(19) United States
(12) Patent Application Publication
(10) Pub. No.: US 2001/0006959 A1
(43) Pub. Date: Jul. 5, 2001

(54) COMPOSTIION, METHOD AND SYSTEM FOR IDENTIFYING NOVEL ANTIMICROBIAL AGENTS

(76) Inventors: TIMOTHY G. PALZKILL, HOUSTON, TX (US); JOSEPH PETROSINO, HOUSTON, TX (US); WANZHI HUANG, HOUSTON, TX (US)

Correspondence Address: MATTHEW E. BURR, 1601 ELM STREET, 3000 THANKSGIVING TOWER, DALLAS, TX, 75201, US

(21) Appl. No.: 09/160,405

(22) Filed: Sep. 25, 1998

(51) Int. Cl. A61K 31/665; A01N 57/00

(52) U.S. Cl. 514/100

(57) ABSTRACT

An composition, method and system for identifying novel antimicrobial agents including the steps of, displaying a β-lactamase inhibitor protein on a virus, contacting the virus with a β-lactamase binding protein target, selecting for the virus that has a higher affinity for the target and testing the β-lactamase inhibitor protein for antimicrobial activity, is disclosed. The invention also includes a nucleic acid encoding a fusion protein comprising a β-lactamase inhibitor protein and an affinity carrier and the protein expressed therefrom. Mutant β-lactamase inhibitor proteins may be produced, characterized, isolated and expressed in prokaryotic cells and used as antimicrobial agents.

The government owns certain rights in the present invention pursuant to grant number A132956 from the National Institutes of Health.

### TECHNICAL FIELD OF THE INVENTION

The present invention relates in general to the field of antimicrobial agents, and more particularly, to the identification, selection and isolation of enhanced antimicrobial agents for use in strains of bacteria resistant to β-lactam containing, and β-lactam related, antibiotics.

### BACKGROUND OF THE INVENTION

Without limiting the scope of the invention, its background is described in connection with penicillin and cephalosporin based antimicrobial agents, as an example.

Heretofore, in this field, β-lactam antibiotics, such as the penicillins and cephalosporins, have been the most often used antimicrobial agents. Because of their widespread use, bacterial resistance to these antibiotics has become an increasing problem (Davies, J., “Inactivation of antibiotics and the dissemination of resistance genes,” Science. 264:375-382 (1994)).

The most common mechanism of resistance is the production of β-lactamases. β-lactamases are generally secreted to the periplasm of gram-negative bacteria (or extracellularly in gram-positive bacteria), where they hydrolyze, and thereby inactivate, the β-lactam ring of these antibiotics. There are a large number of β-lactamases that are found encoded either on plasmids or on the bacterial chromosome (Bush, K., G. A. Jacoby, and A. A. Medeiros, “A functional classification scheme for β-lactamases and its correlation with molecular structure,” Antimicrob. Agents Chemother., 39:1211-1233 (1995)). In gram-negative bacteria, for example, the most common plasmid-based β-lactamase is the TEM-1 β-lactamase.

An effective means of combating TEM-1 β-lactamase mediated resistance has been the clinical use of small molecule β-lactamase inhibitors such as sulbactam and clavulanic acid (Parker, R. H., and M. Eggleston, “β-lactamase inhibitors: another approach to overcoming antimicrobial resistance,” Infect. Control., 8:36-40 (1987)). These molecules, however, do not possess significant antimicrobial activity themselves but are used in conjunction with other β-lactam antibiotics, such as ampicillin. This class of molecules act by protecting the antibiotic from the action of β-lactamase and thereby restore the therapeutic value of the antibiotic. While the combination approach worked for a period of time, new reports of resistance to β-lactam:β-lactamase inhibitor therapy due to mutations in β-lactamase have enabled bacteria to avoid inactivation by the inhibitor while retaining the ability to hydrolyze β-lactam antibiotics (Imtiaz, U., E. Billings, J. R. Knox, E. K. Manavathu, S. A. Lemer, and S. Mobashery, “Inactivation of class A β-lactamases by clavulanic acid: the role of arginine 244 in a proposed nonconcerted sequence of events,” J. Am. Chem. Soc., 115:4435-4442 (1993)).

The need to identify and isolate novel antimicrobial agents is further accentuated by the identification of mutations within β-lactamase that allow it to hydrolyze antibiotics designed to circumvent it's primary activity. In some gram-positive bacteria, such as Streptococcus pneumoniae, resistance to β-lactam antibiotics is acquired by mutations in the penicillin-binding-proteins targeted by the drugs. For example, methicillin-resistant Staphylococcus aureus (MRSA) has acquired the penicillin-binding-protein (PBP) PBP2a, which is able to catalyze the cross-linking of the bacterial cell-wall, but does not bind any β-lactam antibiotics. Many MRSAs are also resistant to other classes of antibiotics as well, and as a result, some MRSA infections are only treatable with the glycopeptide antibiotic vancomycin.

### SUMMARY OF THE INVENTION

It has been found, however, that the present methods for identifying and customizing new antimicrobial agents to drug resistant strains of bacteria are unable to cope with the increase in nosocomial infection that are multiple-drug resistant (MDR). A significant problem of current isolation and identification systems is that they rely on the serendipitous isolation and characterization of antimicrobial agents. Alternatively, rational drug design systems based on the X-ray structure of the target require the structure of the target to be known.

The development of novel β-lactamase and penicillin-binding-protein (PBPs) inhibitors, provides a new way to treat bacterial infections, and in particular, resistance to the β-lactam ring containing antibiotics. As β-lactamases are generally believed to have evolved from PBPs, minor changes in the structure of β-lactamase inhibitor proteins (BLIPs) are expected to create novel inhibitors to PBPs. The present invention may be used not only to isolate novel inhibitors, but also to understand how the amino acid sequence of BLIP encodes its binding affinity for β-lactamases, and to a lesser extent for PBPs. The compositions, methods and system disclosed herein have been used to facilitate the development of novel inhibitors with potent activity for the extended spectrum β-lactamases (ESBLs) and for the PBPs. The identification of the residues involved in inhibition and specificity have been identified, as disclosed herein, and have been targeted for engineering of BLIP mutants with higher and directed inhibitory activity for different β-lactamases and PBPs.

In order to develop new antimicrobial agents based on directed, self-selective mechanisms, the present inventors have recognized that molecules, such as specific β-lactamase inhibitory proteins (BLIP) and peptides, may be isolated and selected for by analyzing β-lactamase binding. For example, the small molecule inhibitor of β-lactamases, clavulanic acid, is a natural product from Streptomyces clavuligerus. In addition to clavulanic acid, S. clavuligerus also produces a protein inhibitor of β-lactamase called β-lactamase inhibitory-protein (BLIP). BLIP is a 165 amino acid protein encoded by the bli gene that binds and inhibits TEM-1 β-lactamase with a reported Kᵢ of 0.6 nM. In addition, BLIP has been reported to inhibit the Enterococcus faecalis PBP5 with a Kᵢ of 12 μM. As BLIP binds to β-lactamases from both gram-negative and gram-positive bacteria (albeit with reduced affinity) the present inventors recognized that BLIP-β-lactamase interactions could be exploited to develop a system to isolate, and improve the affinity of, a novel antimicrobial.

X-ray structure of BLIP has been solved both alone and in complex with TEM-1 β-lactamase to reveal the residues making up the binding surface of BLIP (Strynadka, N. C. J., S. E. Jensen, P. M. Alzari, and M. N. G. James, “A potent new mode of β-lactamase inhibition revealed by the 1.7 Å X-ray crystallographic structure of the TEM-1-BLIP complex,” Nature Struct. Biol. 3:290-297 (1996); and Strynadka, N. C. J., S. E. Jensen, K. Johns, H. Blanchard, M. Page, A. Matagne, J.-M. Frere, and M. N. G. James, “Structural and kinetic characterization of a β-lactamase-inhibitor protein,” Nature, 368:657-660 (1994)). The present inventors realized, however, that the X-ray crystallographic data has failed to lead to rational strategies for drug design, as the TEM-1 β-lactamase has vastly different interaction characteristics from other β-lactamases (Id.).

To overcome these and other problems in the art, the present inventors have expressed BLIP in bacteria as a fusion protein with the g3p coat protein of the M13 bacteriophage. Recombinant bacteriophage expressing the fusion protein are able to bind specifically, and with high affinity, to the TEM-1 β-lactamase. Therefore, the BLIP-g3p fusion protein was expressed and displayed on the surface of the bacteriophage in a folded, functional form.

Display of functional BLIP was accomplished by constructing a new phage display vector that allowed the BLIP protein to be expressed under the control of the constitutive TEM-1 β-lactamase promoter and secreted under the direction of the β-lactamase signal sequence. The low-levels of transcription of the BLIP-g3p fusion are not toxic to E. coli, leading to titers of 1×10¹² to 1×10¹³ phage/ml.

Another problem overcome by the present inventors was the high frequency of frameshift mutations of the BLIP gene found among transformants after insertion of PCR produced BLIP gene into the pG3-C3 vector. To obtain wild-type transformants it was necessary to obtain a non-mutant sequence by a functional selection for BLIP-phage that bound to immobilized β-lactamase. A high frequency of clones contained frameshift mutations in BLIP in the non-selected population, which may be due to a high frequency of polymerase errors during PCR on templates having a high G-C content.

The development of the BLIP-phage system was then exploited to determine which residues on BLIP are critical for TEM-1 β-lactamase binding and to select for variants that bind tightly to other β-lactamases or penicillin binding proteins. To achieve this, libraries of random mutants of BLIP were created in the pG3-BLIP vector. The phage libraries were then panned on purified TEM-1 β-lactamases and other β-lactamases, as described hereinbelow.

More particularly, one embodiment of the present invention is a nucleic acid segment encoding a fusion protein, wherein the segment includes a β-lactamase inhibitor protein and a viral coat protein carboxy from the β-lactamase inhibitor protein. The segment may be incorporated as part of a recombinant vector, and in one embodiment a recombinant expression vector. The segment encoding the fusion protein may expressed in, e.g., E. coli. The segment and recombinant vector may be introduced in a suitable host to express and form virions based on the gene encoding a fusion protein, the fusion protein, which may also include a signal peptide.

Another embodiment of the invention is a fusion protein composition having a β-lactamase inhibitor protein and an affinity carrier. The affinity carrier may be amino- or carboxy- from the β-lactamase inhibitor protein to form a fusion protein. By affinity carrier is meant a polypeptide that confers affinity to the fusion protein distinct from the binding of BLIP to extended spectrum β-lactamases (ESBL). The affinity of the polypeptide may be for a substrate (e.g., maltose binding protein to a maltose affinity column or a histidine tag for nickel or cobalt) or of a separate molecule for the polypeptide (e.g., an antibody against a polypeptide tag such as myc or FLAG). Alternatively, the β-lactamase inhibitor protein can be attached to the surface (e.g., the protein coat) of a virion by chemical conjugation using, e.g., bivalent crosslinkers. The β-lactamase inhibitor fusion protein may be expressed in E. coli and may also include a carrier protein (e.g., maltose binding protein) or a short peptide tag (e.g., a Histidine tag).

Yet another embodiment of the present invention is a method of isolating an antimicrobial agent including the steps of, displaying a β-lactamase inhibitor protein on a virus, contacting the virus with a β-lactamase binding protein target, selecting for the virus that has a higher affinity for the target and testing the β-lactamase inhibitor protein for antimicrobial activity.

The present inventors have used site-directed mutagenesis techniques, and the creation of mutant libraries, to increase BLIP binding and inhibition of β-lactamases and penicillin-binding proteins. Using the tools and the system developed by the present inventors, BLIP can be used as a molecular scaffolding to engineer binding interactions and thereby create new BLIP-based antibiotics and inhibitors. Using the present invention, smaller sized fragments of the BLIP protein, and mutants thereof, may be selected for using the phage display system disclosed herein. The smaller BLIP fragments, and mutants thereof, may reduce the antigenicity of the new β-lactamase antimicrobials and due to the reduced size improve its access to target sites having a microbial infection.

Yet another embodiment of the invention is a system for identifying, selecting and improving an antimicrobial agent including the following steps:

(a) creating a mutant β-lactamase inhibitor protein phage display library;

(b) selecting mutant β-lactamase inhibitor protein phage by comparing one or more characteristics of the mutant β-lactamase inhibitor display phage;

(c) cloning the selected mutant β-lactamase inhibitor protein phage;

(d) conducting mutagenesis on the selected mutant β-lactamase inhibitor protein phage to create a new mutant β-lactamase inhibitor protein phage display:

(d1) evaluating the performance of each β-lactamase inhibitor protein phage by panning for those having one or more antimicrobial characteristics,

(d2) eliminating β-lactamase inhibitor protein phage whose performance is less than a specified performance level, and

(d3) selecting mutants from the mutant β-lactamase inhibitor protein phage, each mutants of mutant β-lactamase inhibitor protein phage having antimicrobial performance that is equal to or greater than the specified performance level; and, if necessary,

(e) repeating steps (b) and (c).

### BRIEF DESCRIPTION OF THE DRAWINGS

For a more complete understanding of the features and advantages of the present invention, reference is now made to the detailed description of the invention along with the accompanying figures in which corresponding numerals in the different figures refer to corresponding parts and in which:

FIG. 1 shows a map of the relative positions and restriction endonuclease cleavage sites of the pG3-BLIP plasmid that encodes a BLIP-g3p fusion protein;

FIG. 2 is a graph showing the results of an ELISA assay for phage binding;

FIG. 3 shows the affinity of the BLIP-phage;

FIG. 4 is a flowchart of the phage display system for isolating novel antimicrobial agents;

FIG. 5 is diagram of a truncated BLIP-g3p fusion protein;

FIG. 6 is an outline of a DNA shuffling procedure for the selection of recombinants.

### DETAILED DESCRIPTION OF THE INVENTION

While the making and using of various embodiments of the present invention are discussed in detail below, it should be appreciated that the present invention provides many applicable inventive concepts which can be embodied in a wide variety of specific contexts. The specific embodiments discussed herein are merely illustrative of specific ways to make and use the invention and do not delimit the scope of the invention.

The display of proteins on the surface of filamentous phage has been shown to be a powerful method to select variants of a protein with altered binding properties from large combinatorial libraries of mutants. The β-lactamase inhibitory protein (BLIP) is a 165 amino acid protein that binds and inhibits TEM-1 β-lactamase-catalyzed hydrolysis of the penicillin and cephalosporin antibiotics. The present inventors have constructed a new phagemid vector and have developed a method of using the vector as part of a system to display or produce BLIP on the surface of filamentous phage. The recombinant BLIP protein was shown to have binding to immobilized β-lactamase. The binding of the recombinant BLIP to β-lactamase was specific as it can be competed off by the addition of soluble β-lactamase.

A two-step phage ELISA procedure was also used to demonstrate that the BLIP-displaying phage bind β-lactamase with an IC₅₀ of 1 nM, which compares favorably with a previously published Kᵢ of 0.6 nM. Therefore, the present inventors have developed the tools necessary to accompany a system for protein engineering of BLIP to expand its binding range to other β-lactamases and penicillin binding proteins.

To amplify and select for specific high β-lactamase binders, phage display isolation was used to increase the amount and level of molecular interactions between the potential inhibitor and the β-lactamase. Phage display has proven to be an effective methodology for the selection of binding partners of high affinity or altered specificity.

Briefly, monovalent display of a protein of interest may be achieved by fusing the gene encoding the protein to the N-terminus of the M13 gene III coat protein. In the present invention, high affinity variants are obtained by creating phage libraries containing mutants of the protein of interest and sequencing the corresponding DNA packaged in the phagemid particles after several rounds of binding selection.

The first step in the phage display system is the cloning of the BLIP gene into a new phagemid vector. The vector encodes chloramphenicol resistance and contains an amber codon at the 5′ end of gene III. BLIP is fused at its N-terminus to the β-lactamase signal sequence and at its C-terminus to the protein encoded by gene III of the phage (g3p). Transcription of the fusion is controlled by insertion upstream from the fusion protein of the constitutive β-lactamase promoter. Specific binding of phage containing the BLIP-g3p fusion to immobilized β-lactamase is demonstrated to occur with nanomolar affinity, and has been used to engineer new BLIP binding specificities.

Bacterial Strains

Escherichia coli strain XL1-Blue [F′::Tn10 proA⁺B⁺lacI^{q} Δ(lacZ) M15/recA1 endA1 gyr96(Nal^{r}) thi hsdR17 (r⁻m⁺) supE44 relA1 lac] was used for transformations of ligations and E. coli TG1 [F′ traD36⁺lacI^{q} Δ(lacZ) M15 proA⁺B⁺/supE Δ(hsdM⁻mcrB⁻) (r^{-—}McrB⁻) thi Δ(lac⁻proAB) was used for production, amplification, and titering of bacteriophage.

The phagemid encoding the BLIP-g3p fusion was constructed by inserting a 1365 bp XbaI-BamHI fragment containing gene III from phagemid phGHamg3 (Lowman, H. B., S. H. Bass, N. Simpson, and J. A. Wells, “Selecting high-affinity binding proteins by monovalent phage display,” Biochemistry, 30:10832-10838 (1991)) into XbaI-BamHI digested pBCKS+ (Stratagene, U.S.A.) to create plasmid pG3-CMP. The pG3-CMP plasmid was digested with SalI, the 5′ overhangs were filled-in with Klenow polymerase and dNTPs, and the ends were religated to destroy the SalI site and create plasmid pG3-C2.

A construct containing the coding sequence of TEM-1 β-lactamase fused to gene III (g3p) of M13 was created by PCR amplification of the bla_{TEM-1} gene from plasmid pBG66-N78 (Huang, W., J. Petrosino, M. Hirsch, P. S. Shenkin, and T. Palzkill, “Amino acid sequence determinants of β-lactamase structure and activity,” J. Mol. Biol. 258:688-703 (1996)). This plasmid contains a bla_{TEM-1} gene with a SalI site inserted at nucleotide position 284 of the published sequence (Sutcliffe, J. G., “Nucleotide sequence of the ampicillin resistance gene of Escherichia coli plasmid pBR322,” Proc. Natl. Acad. Sci. USA., 75:3737-3741 (1978)), which is the codon for the third amino acid position beyond the cleavage site of the β-lactamase signal sequence. Therefore, the SalI site may be used to fuse other genes behind the promoter and signal sequence of β-lactamase. The bla_{TEM-1} was amplified using primers containing internal restriction sites, as will be known to those of skill in the art in light of the published sequences.

Briefly, one primer PD-bla1 contains a SacI site, while a second primer PD-bla2 contains an XbaI site. The PCR reaction was performed using the Advantage cDNA PCR kit (Clontech, Inc., U.S.A.) using buffer conditions recommended by the manufacturer. The reactions were cycled 30 times at 94° C. for 1 min. followed by 64° C. for 4 min. After cycle 30 the reactions were incubated at 64° C. for 10 min, the PCR product was digested with SacI and XbaI and inserted into SacI-XbaI digested pG3-C2 plasmid to create pG3-C3. The SacI-XbaI fragment of pG3-C3 contains approximately 150 bp of sequence upstream of the bla_{TEM-1} gene and therefore contains the constitutive β-lactamase promoter. The XbaI site is the point of fusion between β-lactamase and gene III (g3p). There is an amber codon at the fusion site and so the fusion protein will only be made in an amber-suppressor strain of E. coli.

S. clavuligerus genomic DNA was used as template for amplification of the bli gene. S. clavuligerus (ATCC 27064) cultures were grown in TSA broth containing 1% starch for 64 hours. Chromosomal DNA was isolated using the Puregene kit (Gentra) for Gram-positive bacteria. The bli gene was amplified using PCR primers, the 5′ primer BLIPXHOI contains a XhoI site and the 3′ the primer BLIPXBAI contains an XbaI site. The primers were designed to amplify only the mature portion of BLIP which includes codons 37 to 165 of the bli gene (Doran, J. L., B. K. Leskiw, S. Aippersbach, and S. E. Jensen, “Isolation and characterization of a β-lactamase-inhibitory protein from Streptomyces clavuligerus and cloning and analysis of the corresponding gene,” J. Bacteriol., 172:4909-4918 (1990)). The PCR conditions were identical to those described above for the bla_{TEM-1} gene. The BLIP PCR product was purified using a QIAquick PCR Purification kit from Qiagen (Qiagen, U.S.A.) following the instructions of the manufacturer. The purified product was digested with XhoI and XbaI and gel-purified on a SeaPlaque low melt agarose gel. The pG3-C3 plasmid was digested with SalI and XbaI to release the β-lactamase gene. The remainder of the vector was purified from the released β-lactamase gene by low melt agarose.

FIG. 1 shows a map of the relative positions and restriction endonuclease cleavage sites of the pG3-BLIP plasmid that encodes and expresses a BLIP-g3p fusion protein of, and for use with, the invention. The XhoI-XbaI digested BLIP fragment is inserted into the SalI-XbaI digested of the pG3-C3 vector, described hereinabove, to create a fusion of the β-lactamase signal sequence fused to the N-terminus of BLIP and g3p at the C-terminus of BLIP. The BLIP-g3p fusion containing pG3-BLIP plasmid was electroporated into E. coli XL1-Blue and individual colonies were screened for BLIP inserts by DNA restriction enzyme analysis.

E. coli RB791 (Strain W3110 lacI^{qL8}) (available from ATCC) was used to express BLIP and the D49A and F142A BLIP mutants. Plasmid pTP123 is a cmp^{r} amp^{r} derivative of pTrc 99A (Pharmacia, Sweden). It was created by ligating the SmaI cassette from pKRP10 into BsaI and XmnI digested pTrc 99A. The BsaI and XmnI sites were filled in using the Klenow fragment of DNA Polymerase I prior to ligation. This cloning step inserts a chloramphenicol acetyl transferase (cat) gene into the rmBT₁T₂ transcriptional terminators and part of the β-lactamase gene encoded by pTrc 99A. As a result, functional β-lactamase is not expressed, and potential difficulty in BLIP purification due to binding of endogenous β-lactamase is avoided. The cat gene in TP123 is in the same orientation as the trc promoter.

A 6X Histidine (6XHis) tag was first inserted between the β-lactamase signal sequence and the BLIP coding sequence of pG3-BLIP by overlapping PCR mutagenesis. A SacI site in PD-bla1 and a XbaI site in MALBLI-2 allowed the PCR product to be cloned into SacI and XbaI digested pTP123 following treatment of the vector with calf-intestinal alkaline phosphatase (CIP). The final SacI/XbaI fragment contains, from 5′ to 3′, the β-lactamase constitutive promoter, the β-lactamase periplasmic signal sequence, and a BLIP N-terminus 6XHis tag followed by the mature BLIP coding sequence. The sequence of this clone was confirmed by the dideoxy-chain termination method, and was named pGR32. The positioning of this construct in pTP123 allows N-terminal His-tagged BLIP to be expressed either under the β-lactamase constitutive promoter, or by induction of the trc promoter with IPTG. The 6XHis tag facilitates the purification of BLIP using an appropriate nickel or cobalt based affinity column, while the β-lactamase signal sequence enables BLIP to be transported to the periplasmic space, thus eliminating the need to isolate whole cell extracts for BLIP purification.

PCR Mutagenesis

Construction of the BLIP D49A and F142A mutants was accomplished by overlapping PCR mutagenesis. PD-bla1 and MALBLI-2 were used as external primers in these mutagenesis reactions. PD-bla1 and MALBLI-2 were used to amplify the full-size mutagenized product. Both mutagenic PCR products were cloned into SacI/XbaI digested and CIP treated pTP123. The D49A mutant was named pJP128, and the F142A mutant was named pJP129. The DNA sequence of each mutant was confirmed by the dideoxy-chain termination method.

BLIP and β-lactamase Expression and Purification

Plasmid pGR32, pJP128, and pJP129 were transformed into E. coli RB791 by electroporation. An overnight culture of each was grown shaking in 40 mL Luria-Bertani (LB) medium at 37 C. in the presence of 12.5 μg/mL chloramphenicol. The 40 mL of overnight culture were used to inoculate 2 L of LB media containing 12.5 μg/mL chloramphenicol. The bacteria then grown shaking at 25 C. until OD₆₀₀=1.2. For induction of BLIP, 3 mM IPTG was added to each culture, and the cultures were then allowed to grow an additional 5 hours.

Following the 5 hour induction, the cells were pelleted and resuspended in 15 mL sonication buffer (20 mM Tris-HCl (pH 8.0) and 500 mM NaCl). The cells were then sonicated in two batches, and insoluble material was pelleted by centrifugation. The soluble protein in the supernatant was purified over a 4 mL TALON column (Clontech) according to the manufacturer's instructions. A 4 mM imidizole wash step was utilized to remove protein from the column which bound less tightly than the His-tagged BLIP. BLIP was eluted using an elution buffer consisting of 50 mM imidizole added to the sonication buffer (pH 8.0). Fractions were examined by SDS-PAGE to estimate purity and yield. Approximately 500 μg of >90% pure BLIP could be isolated for every two liters of culture using this strategy. Wild-type β-lactamase and the G238S and E104K extended-spectrum mutants were expressed and purified as previously described.

Phage Preparation and Panning

After overnight growth, E. coli cells were removed by centrifugation and the phage were precipitated from the supernatant with 1/5 volume of 20% PEG, 2.5 M NaCl. The phage were pelleted by centrifugation and resuspended in 1/100 of the original culture volume of STE (0.1 M NaCl, 10 mM Tris-Cl pH 8.0, 1 mM EDTA pH 8.0). The phage titer was determined by making serial dilutions of 0.1 ml total volume and adding 0.2 ml of E. coli TG1 cells. Aliquots of 0.15 ml were plated on LB agar supplemented with 12.5 μg/ml of chloramphenicol. After overnight growth at 37° C., the number of colonies was determined and the titer was calculated.

β-lactamase was immobilized for panning by covalent attachment to 1-μm oxirane-acrylic beads (Sigma, U.S.A.). Briefly, 50 mg of beads was suspended in 0.1 M sodium carbonate buffer (pH 8.6) and was incubated with purified TEM-1 β-lactamase at a concentration of 0.04 mg/ml for 24 hrs at 4° C. Unreacted oxirane groups were blocked by incubating with 10 mg/ml bovine serum albumin (BSA) overnight at 4° C. The beads were then pelleted and washed several times with buffer A which is Tris-buffered-saline containing 1 mg/ml BSA and 0.5 g/l Tween 20. The TEM-1 containing beads were stored in buffer A in a final volume of 0.5 ml.

For panning, 1×10¹¹ phage were contacted with 5 mg of β-lactamase conjugated oxirane beads in a final volume of 0.5 ml in buffer A. The phage-β-lactamase bead mixture was incubated for 2 hrs at room temperature with rocking to reach equilibrium. The phage-β -lactamase beads were then washed 10 times with 0.75 ml of buffer A. The bound phage may be eluted from the phage-β-lactamase beads by incubation with, e.g., 0.2 ml of elution buffer (0.1 M glycine pH 2.2, 1 mg/ml BSA, 0.5 g/l Tween 20, 0.1 M KCl) for 30 minutes. Those of skill in the art will recognize that other elution buffers may be used to release the phage from the β-lactamase beads. Elution buffers may or may not affect phage viability, where they do, the phage DNA may isolated and repackaged into new virions.

The elution mixture may be neutralized with 25 μl of 1 M Tris-Cl pH 8.0. The phage titer of the elution mixture was determined as described above. The eluted phage were amplified by adding 0.15 ml of the neutralized elution mixture to 5 ml of E. coli TG1 cells. After 30 minutes incubation at room temperature, 25 ml of 2YT medium was added along with 1×10⁹ VCS M13 helper phage (Stratagene, U.S.A.). The phage were precipitated as described above after overnight incubation with shaking at 37° C.

BLIP Inhibition Assay

Varying concentrations of BLIP were incubated with 1 nM β-lactamase for 2 hours at 25 C. In the G238S studies 2 nM of the β-lactamase were used. The enzyme-inhibitor incubation was conducted in 0.05 M phosphate buffer (pH 7.0) containing 1 mg/mL bovine serum albumin (BSA). Following the 2 hour incubation, cephaloridine was added at a concentration of at least 10-fold lower than the Kₘ of the β-lactamase being tested (e.g. wild-type TEM-1 β-lactamase has a Kₘ approximately 700 μM for cephaloridine, therefore 70 μM cephaloridine was added to the TEM-1/BLIP incubation). The final volume for the reaction was 0.5 mL. Hydrolysis of cephaloridine was monitored at A₂₆₀ on a Beckman DU70 spectrophotometer. The extinction coefficient used for cephaloridine was Δε=10,200 M⁻¹cm⁻¹. Plots of the concentration of free β-lactamase vs. inhibitor concentration were fit by nonlinear regression analysis to Equation 1, where Vᵢ/V₀ is the fractional β-lactamase activity (steady state inhibited rate divided by the uninhibited rate), [E₀] is the total β-lactamase active site concentration, and [I₀] is the total inhibitor concentration. From the equation, apparent equilibrium dissociation constants (Kᵢ*) were determined.

Vᵢ/V₀=[E₀]+[I₀]+Kᵢ* sqrt (([E₀]+[I₀]+Kᵢ*)2−(4[E₀][I₀])/2[E₀] (Equation 1)

Phage ELISA

A two-step phage ELISA was performed to measure BLIP-phage affinity for TEM-1 β-lactamase. Microtiter plates (Nunc, U.S.A., Maxisorp, 96 well) were coated with purified TEM-1 β-lactamase (at 10 μg/ml) in 50 mM sodium carbonate (pH 9.6) at 4° C. overnight. The plates were then blocked with SuperBlock (Pierce) for 2 hours at room temperature. Serial dilutions of the BLIP phage stock were added to the wells and incubated for 2 hours at room temperature in buffer A at a final volume of 0.15 ml. After washing the plates several times with buffer A, the bound phage were probed with a sheep anti-M13 polyclonal antibody conjugated to horseradish peroxidase. To determine phage affinity, serial dilutions of β-lactamase and a subsaturating concentration of 1.3×10¹¹ BLIP phage were added to wells in 0.1 ml of buffer A. After 2 hrs at room temperature the wells were washed multiple times with buffer A and bound phage were probed as described above.

To identify the wild-type BLIP sequence, approximately 10,000 colonies were pooled after transformation of the ligation mix of the BLIP PCR fragment with the pG3-C3 vector. Helper phage was added to the pooled colony culture and phage were isolated. The phage were bound to oxirane beads conjugated to β-lactamase, washed extensively, and then eluted with a low pH buffer as described in Materials and Methods. The phage isolated in this manner were predicted to contain a functional BLIP fusion protein. DNA sequence analysis of four clones from the elution identified one clone with the wild-type sequence and three clones with a D163N substitution near the C-terminus of BLIP. The clone with the wild-type sequence was further characterized.

A two-step phage ELISA method was used to demonstrate that BLIP is functionally displayed on the surface of the bacteriophage and that the phage bind specifically to β-lactamase. Purified TEM-1 β-lactamase was coated onto ELISA plates and serial dilutions of phage displaying wild-type BLIP were allowed to bind to the immobilized protein in the presence of a large excess of bovine serum albumin (BSA). After washing, bound phage were stained with HRP-conjugated anti-M13 antibody.

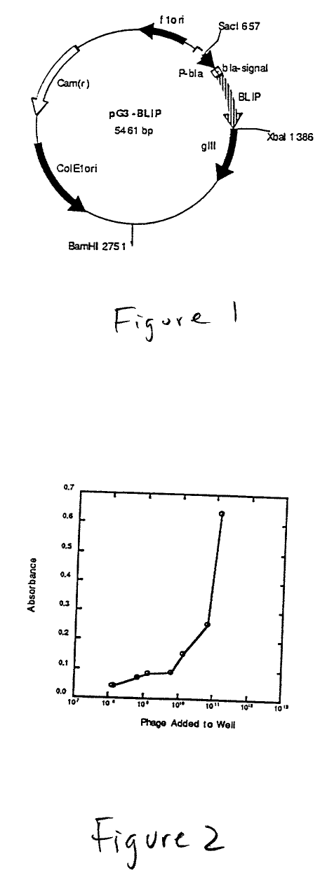

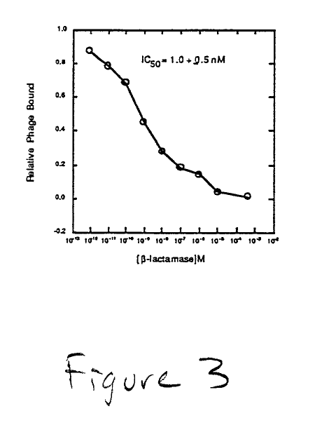

FIG. 3 is a graph showing the relative optical density of the expressed BLIP-g3p fusion protein on the surface of chimeric phage in the phage ELISA assay. To quantitate binding, β-lactamase was coated onto ELISA plates and a constant subsaturating concentration of pG3-BLIP phage was added with serial dilutions of purified β-lactamase. The binding curve of FIG. 2 was used to determine the affinity of BLIP-g3p phage to TEM-1 coated ELISA plates at subsaturating concentration of phage (1.3×10¹¹ phage). The IC₅₀ value given shows the concentration of competing β-lactamase that results in half-maximal binding to the phagemid. The half-maximal concentration was calculated by converting the data in FIG. 3 from log to linear values and fitting the binding curve to the equation for a hyperbola. Affinities (IC₅₀) were calculated as the concentration of competing β-lactamase that resulted in half-maximal BLIP phage binding. As seen in FIG. 3, the pG3-BLIP phage were competed off of the immobilized β-lactamase with soluble β-lactamase at an IC₅₀ of 1 nM. This affinity compares favorably to the published Kᵢ value of 0.6 nM for the BLIP-β-lactamase interaction. Therefore, the inventors have expressed BLIP on the surface of the bacteriophage in a form that binds tightly and specifically to β-lactamase.

The next step in the antimicrobial isolation system is to specifically enrich for BLIP-phage by panning on β-lactamase. In order to use the pG3-BLIP phagemid to engineer the BLIP protein for altered binding properties it is necessary to be able to select binding phage by panning on an immobilized substrate. This was demonstrated by attaching purified β-lactamase to oxirane-acrylic beads and incubating the beads with 1×10¹¹ phage from the pG3-BLIP phage stock in the presence of a large excess of BSA.

Two controls were performed to demonstrate that phage binding to the beads was dependent on the BLIP-β-lactamase interaction. First, an internal control was performed by adding 1×10¹¹ phage that did not display BLIP along with the 1×10¹¹ pG3-BLIP phage to the oxirane beads conjugated to β-lactamase. The phagemid, pG3-SPT, which produced the non-displaying phage was constructed by inserting a gene cassette encoding spectinomycin resistance (Reece, K. S., “New plasmids carrying antibiotic-resistance cassettes,” Gene, 165:141-142 (1995)) into the chloramphenicol resistance gene of pG3-C2.

One advantage in the design of this antimicrobial agent selection system is that the extent of enrichment of non-displaying phage versus BLIP-displaying phage may be determined simply by titering the phage recovered from the oxirane beads on spectinomycin-containing agar plates as well as chloramphenicol-containing agar plates. The results in Table 1 illustrate that 27-fold more pG3-BLIP phage were recovered from the β-lactamase beads than non-displaying phage when no soluble β-lactamase is added to compete for binding to the pG3-BLIP phage (compare pG3-BLIP versus pG3-SPT in bla beads+0 μM bla column).

TABLE I

| | | | | | |
|---|---|---|---|---|---|
| Phage | bla^{a}beads + | bla beads + | bla beads + | bla beads + | BSA beads |
| | 0 μM bla | 0.1 μM bla | 1 μM bla | 10 μM bla | |
| pG3-BLIP | 2.3 × 10^{6b} | 3.4 × 10⁵ | 2.7 × 10⁵ | 1.9 × 10⁵ | 1.8 × 10⁵ |
| pG3-SPT | 8.6 × 10^{4b} | 9.9 × 10⁴ | 6.9 × 10⁴ | 6.7 × 10⁴ | 8.8 × 10⁴ |

^{a}bla represents β-lactamase
^{b}The number of phage particles eluted from beads after the application of 1 × 10¹¹phage in the binding reaction. Note that each column represents a binding experiment and in each experiment 1 × 10¹¹of pG3-BLIP phage and pG3-SPT phage were added simultaneously to the beads.

As increasing amounts of soluble β-lactamase was added the enrichment of pG3-BLIP phage over non-displaying phage decreased to 3-fold when 10 μM soluble β-lactamase was added (bla beads+10.0 μM bla column). Therefore, soluble β-lactamase is able to compete off the binding of pG3-BLIP phage to the β-lactamase coated oxirane beads. These data show that BLIP phage was specifically enriched by a round of panning against immobilized β-lactamase.

For the second control, 1×10¹¹ pG3-BLIP phage along with 1×10¹¹ non-displaying pG3-SPT phage were incubated with oxirane beads to which only BSA had been attached. The data in Table 1 indicate that 13-fold more BLIP-phage were bound to β-lactamase than to the BSA control (compare pG3-BLIP in the bla beads+0 μM bla versus BSA beads columns). In addition, there was only a 2-fold difference in the number of BLIP-phage versus non-displaying phage recovered from the BSA beads (compare pG3-BLIP and pG3-SPT in the BSA beads column). This is in contrast to the 27-fold difference between pG3-BLIP phage and non-displaying phage recovered from β-lactamase beads described above. These data show that BLIP phage bind specifically to immobilized β-lactamase.

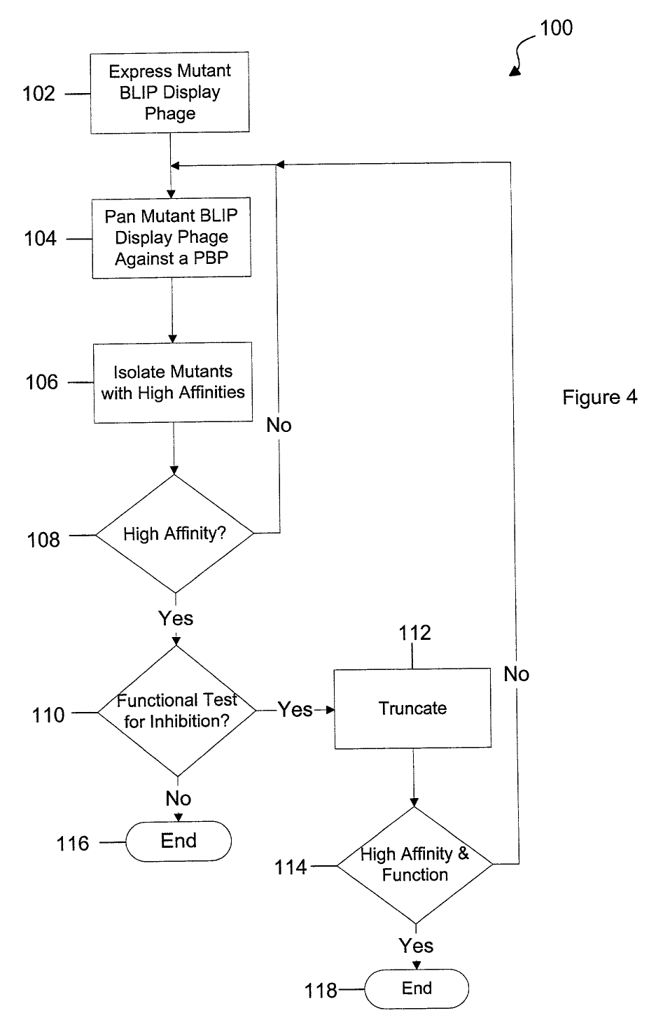

FIG. 4 is a flowchart 100 showing the basic steps of the system for identifying and isolating β-lactamase and β-lactam binding protein inhibitors using the phage display system disclosed herein. In step 102, a library of mutant BLIP derived proteins are displayed on phage particles (mBLIP-PP), as disclosed hereinabove. The library may be pooled library that is resuspended in binding buffer. The library of mBLIP-PP are then panned against β-lactamase or β-lactam binding protein (hereinafter “target”) from β-lactamase resistant strains of gram positive or negative bacteria in step 104. In fact, a specific inhibitor may be isolated against a specific strain of a β-lactamase resistant bacteria using the system disclosed herein, thereby customizing the treatment to that specific form of the target.

In step 106, the BLIP-PP variants that have the highest affinity for the target on the panning surface are isolated by causing the phage particles to detach from the target. For mBLIP-PP strains with binding that is insensitive to, e.g., release under acidic conditions, the mBLIP-PP can be denatured and the phage DNA isolated and repackaged into new phage particles. The isolated mBLIP-PP may then be tested to determine their specific binding kinetics to select for high affinity (step 108) and/or tested for functional inhibition of bacterial resistance (step 11). If the mBLIP-PP isolate has functional activity it may be isolated and used for treatment.

Alternatively, the isolated mBLIP-PP can undergo a series of truncations in step 112. Truncation may lead to a decrease in affinity and functional activity, therefor, the isolated and truncated mBLIP-PP can undergo a subsequent round of reselection (steps 104-110) to isolate truncated mBLIP-PP having high affinity or functional activity (step 114). Generally, it is expected that mBLIP proteins having high affinity will also have functional activity against bacterial resistance, but cases may arise in which that is not the case. If the mBLIP protein does not have high affinity or functional activity then end steps 116 and 118 are reached.

The flowchart of FIG. 4 provides a general outline of the mutant selection and improvement system of the present invention. The system may be used to identify, select and even improve antimicrobial agents based on BLIP. The system includes creating a mutant β-lactamase inhibitor protein phage display library using, e.g., DNA shuffling (described hereinbelow). Next, mutant BLIP phage are selected by comparing one or more characteristics of the mutant β-lactamase inhibitor display phage. The characteristics may include antimicrobial activity of the isolated BLIP mutant or affinity to β-lactamases or PBPs. Mutant BLIP phage are cloned and a further round of mutagenesis is conducted on the selected mutant BLIP phage to create a new mutant β-lactamase inhibitor protein phage display library of mutant BLIPs. The performance of each β-lactamase inhibitor protein phage is then evaluated by panning for those having higher affinity for the β-lactamase(s) or PBP(s) used for screening the mutants. Another method of screening may be, for example, a screen for antimicrobial characteristics, wherein the mutant BLIP phage are exposed to bacteria for a period of time sufficient for those having a higher affinity to bind, but not those having a lower affinity.

Next, the BLIP phage whose performance is less than a specified performance level are eliminated, and mutants from the mutant β-lactamase inhibitor protein phage are selected. The mutants of mutant β-lactamase inhibitor protein phage having antimicrobial performance that is equal to or greater than the specified performance level are used for further evaluation and repeated rounds of evolution.

### EXAMPLE 1

### Construction of a Minimal Functional BLIP Protein

BLIP is a 165 amino acid protein that is organized into two domains. The domains have a similar structure suggesting that the protein evolved by a tandem duplication of a single binding domain. Because BLIP uses both of its domains to bind a monomer of β-lactamase, each domain makes unique binding contacts. Based on the structure of the BLIP-β-lactamase co-crystal, domain 1 of BLIP appears to makes 75% of all of the interactions between the proteins.

The present inventors recognized that it would be possible to express BLIP domain 1 independent of domain 2 by truncating the protein after residue 78. The BLIP protein was truncated at residue 78 as a pG3-BLIP phage display vector. The truncation results in domain 1 being fused to the gene III protein for display on the surface of M13 bacteriophage. The truncation was made by PCR of the bli gene as described above for the pG3-BLIP construct except the PCR primer containing the XbaI site for fusion to gene III was designed to amplify the region ending at residue 78 rather than 165.

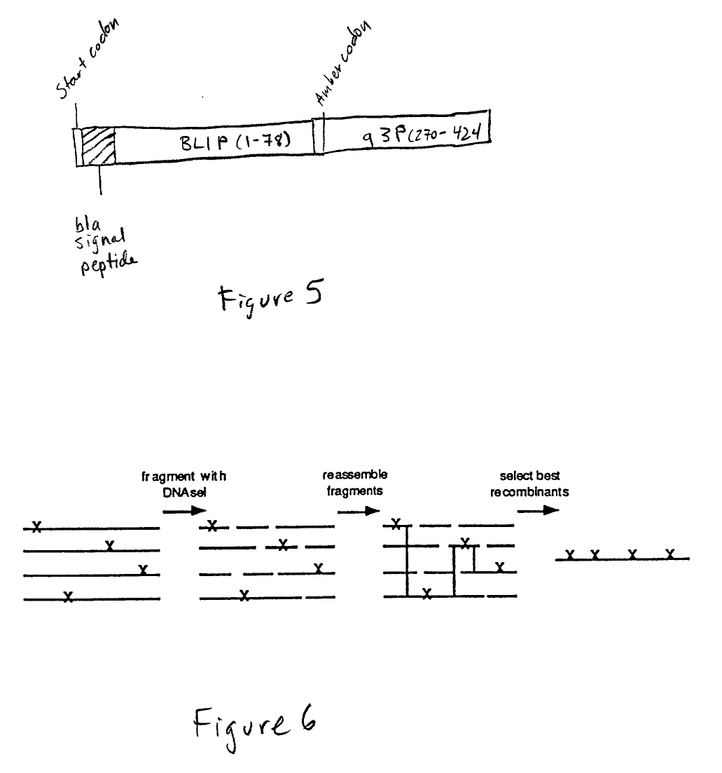

FIG. 5 is a schematic diagram of the N-terminal fusion of BLIP78 to the signal sequence of β-lactamase and the C-terminal fusion after residue 78 of BLIP to the gene III protein of M13.

Phage ELISA experiments confirmed that the truncated protein is functional in β-lactamase binding. Briefly, soluble β-lactamase was used to coat the wells of a 96-well ELISA plate. The wells were blocked and phage displaying the BLIP78 truncated protein were allowed to bind the immobilized β-lactamase. The binding reactions were washed extensively, and bound phage were detected with an HRP-conjugated anti-M13 antibody. As a positive control, an equivalent number of phage that displayed the wild-type BLIP protein were also tested for binding. As a negative control, phage displaying the wild-type BLIP protein were allowed to bind a well that had been coated with BSA. The results indicated that the phage displaying BLIP78 bound β-lactamase only 6-fold less efficiently than phage displaying wild-type BLIP. The BLIP78 phage also bound the immobilized β-lactamase at levels 10-fold higher than background, suggesting that BLIP78 is functional. The weaker but detectable binding of BLIP78 to β-lactamase may result from the loss of interactions involving domain 2 or it may reflect a loss in the stability of domain 1 in the absence of domain 2 or a combination of these effects.

Next, mutants of the truncated BLIP78 that had wild-type, or better, binding to immobilized β-lactamase were isolated using the system as disclosed herein. Multiple rounds of mutagenesis and binding selection were used to evolve the BLIP78 protein back into a tight binder of β-lactamase. The directed evolution method that was employed was DNA shuffling, which produces high-frequency recombination and reassortment of mutations in sequences that have been selected for binding.

FIG. 6 shows the general outline of the DNA shuffling method used to create mutants of mutants (e.g., truncated BLIP) for the selection of mutants having higher affinity for a particular β-lactamase or PBP. Briefly, binding BLIP78 phage, for example, were eluted and amplified, and panning repeated. After elution of phage from the second round of panning, the BLIP78 gene was amplified from the pool by PCR. The PCR primers were removed (e.g., using a Qiagen spin column), and the product was digested with DNASEI to randomly fragment the DNA into fragments less than 100 bp in size. The fragmented DNA was reassembled into full-sized BLIP78 genes by performing PCR in the absence of outside primers. The small fragments self-prime each other during each round of cycling and, after multiple cycles, full-sized genes are obtained as depicted in FIG. 6. The DNA shuffling procedure accelerates the evolution of β-lactamase binding mutants by allowing recombination to occur between genes that were selected for binding in the previous round. DNA shuffling also permits mutations that are far apart in the primary sequence to be combined in a single molecule. Additionally, the PCR amplification steps in the DNA shuffling protocol also lead to the introduction of new random mutations that add diversity to the population.

The BLIP78 gene was amplified by PCR from the pG3-BLIP78 phagemid using Taq polymerase. The PCR product was purified and reinserted into the phage display plasmid. A total of 123,000 colonies were pooled and helper phage was added to produce a collection of phage displaying mutant derivatives of BLIP78. To select binding mutants, the phage were panned on oxirane acrylic beads conjugated to β-lactamase. The reassembled BLIP78 product was inserted into the pG3-BLIP78 plasmid. A total of 1.42×10⁶ colonies were pooled and helper phage was added to create a phage population displaying variant BLIP78 proteins. The phage population displaying the BLIP78 variant proteins was panned on immobilized β-lactamase for two rounds to select the tightest binding mutants, The process of DNA Shuffling was then repeated and tight binders were again selected by two rounds of panning.

After the second round of DNA shuffling, individual clones from the β-lactamase binding selection were isolated by infecting E. coli with the phage eluted from the immobilized β-lactamase. The DNA sequence of four clones was determined from which two unique sequences were discovered. Phage ELISA experiments show conclusively that both of these mutants interact with β-lactamase more efficiently than the unmutated BLIP78.

The D49G substitution found in the BLIP78s1 mutant is at a position in BLIP that directly interacts with the active-site of β-lactamase. In contrast, neither the A65T substitution from BLIP78s1, nor the G33S substitution from BLIP78s2, are at positions that make a direct contact with β-lactamase. In addition, further rounds of DNA shuffling and phage panning may be used to obtain a BLIP78 variant that binds this or any other β-lactamase. The compositions, methods and system disclosed herein were used to characterize, isolate and improve upon, the structure and binding characteristics of a tight-binding, minimized BLIP without the need to identify the X-ray structure of a particular β-lactamase or PBP. Furthermore, the compositions, methods and system of the present invention were used to isolate BLIP variants that would not have been predicted to aid in binding and inhibition even with the availability of X-ray structure and predictable protein folding design.

### EXAMPLE 2

### Targeted Mutation and Isolation of β-lactamase Inhibitors

The present inventors realized from the TEM-1/BLIP co-crystal that two BLIP residues, D49 and F142, mimic interactions made by Penicillin G (PenG) when bound in the active site of the β-lactamase TEM-1. To determine the importance of these two residues, the heterologous expression system described hereinabove established for BLIP in E. coli, along with site-directed mutagenesis, was used to change D49 and F142 to alanine. The inhibitory activity of both mutants was examined. It was found that both mutations decrease BLIP inhibitory activity approximately 100-fold with TEM-1 β-lactamase.

To address how these two positions effect specificity, the inhibitory activity of wild-type BLIP, as well as the D49A and. F142A mutants, was determined for two extended-spectrum β-lactamases (the G238S and the E104K TEM variants). Interestingly, the three BLIP proteins inhibited the G238S β-lactamase mutant to the same degree that they inhibited TEM-1. BLIP has a higher Kᵢ for the E104K β-lactamase mutant, suggesting that interactions between BLIP and β-lactamase residue E104 are important for wild-type levels of BLIP inhibition. Substitution of a phenylalanine at position 142 of BLIP, which interacts with the glutamic acid at position 104 of TEM-1 β-lactamase, did not substantially reduce BLIP inhibition of the E104K enzyme as observed with the TEM-1 and G238S β-lactamases. Therefore, the specific BLIP F142/β-lactamase E104 interaction appears essential for wild-type BLIP inhibitory levels.

BLIP from Streptomyces clavuligerus ATCC 27064 was cloned into pG3-cmp to form pG3-BLIP, as described hereinabove. This vector enabled BLIP to be expressed as a fusion to the M13 gene III protein and subsequently displayed on the surface of M13 bacteriophage. The construct is expressed under the constitutive β-lactamase promoter, and also possesses the β-lactamase signal sequence fused to the N-terminus of BLIP. Induction is not necessary for expression, and the fusion protein is transported to the periplasm as evidenced by the proper formation of phage displaying BLIP. In the construction of pGR32, an N-terminal 6-histidine tag is inserted between BLIP and the β-lactamase signal sequence from the pG3-BLIP construct. The SacI/XbaI fragment containing the β-lactamase promoter and signal sequence, the 6xHis tag, and the BLIP coding sequence was then cloned into pTP123, as described hereinabove, so that expression may be directed under the β-lactamase constitutive promoter or the IPTG-inducible trc promoter.

Several growth conditions and IPTG concentrations were used for optimal expression of BLIP. It was found that growth at 25° C., and addition of 3 mM IPTG to E. coli RB791 cells harboring the pGR32 plasmid increased expression of BLIP above background levels. The 6xHis-tag allows BLIP, as well as the D49A and F142A BLIP mutants, to be purified to greater than 90% homogeneity using a TALON affinity column (Clontech, U.S.A.). Concentrations of BLIP were first determined by the method of Bradford, and then quantitative amino acid analysis was performed to confirm the Bradford results (Protein Core Facility—Baylor College of Medicine).

Wild-Type BLIP Kinetics

To determine if the histidine tag affected BLIP inhibitory activity, and to analyze the activity of the BLIP mutants for TEM-1, the ESBLs, and SHV-1 β-lactamase, an inhibitor assay was developed using the cephalosporin cephaloridine. Wild-type or mutant BLIP was incubated with a target for two hours after which cephaloridine (at a concentration 10-fold less than the cephaloradine Kₘ for the β-lactamase being tested) was added. Monitoring the hydrolysis of cephaloridine was used to determine the concentration of uninhibited β-lactamase. Free β-lactamase was calculated as the ratio of cephaloridine activity in the presence of a given quantity of BLIP versus cephaloridine activity in the absence of BLIP. Fitting the data obtained when incubating varying concentrations of wild-type, his-tagged BLIP with 1 nM TEM-1 β-lactamase resulted in a Kᵢ of 0.11 nM. The value returned compares favorably with the previously reported value of 0.6 nM found with BLIP purified from Streptomyces clavuligerus, and suggests that the N-terminal 6xHis tag has little effect on the binding of the inhibitor to the TEM-1 enzyme.

The affinity of wild-type BLIP was then determined for an extended spectrum β-lactamase, the Kᵢ of BLIP for two representative ESBLs was determined. The G238S β-lactamase mutation is the only substitution found in TEM-19, and is also found in many extended spectrum enzymes. This single mutation increases activity for the third generation cephalosporins: ceftazidime and cefotaxime, approximately 70-fold and 40-fold respectively. The E104K mutation, likewise, has been found in many extended-spectrum β-lactamase variants. This mutation increases the activity of β-lactamase approximately 50-fold for ceftazidime and 10-fold for cefotaxime. Wild-type BLIP was found to have a Kᵢ of 0.07 nM for G238S, and a Kᵢ of 138.5 nM for E104K. These values show that the G238S mutation has little effect on wild-type BLIP binding, while the E104K mutation interferes with binding in such a way that the Kᵢ increases 1000-fold. The fact that BLIP has binding affinity for both of these ESBLs is used in the selection system disclosed herein to screen and engineer BLIP mutants for improved binding and inhibition of these β-lactamase.

Mutant BLIP Kinetics

The crystal structure of the BLIP/TEM-1 β-lactamase inhibitory complex shows that D49 and F142 are two amino acids in the inhibitor which mimic domains of the β-lactam PenG when bound to β-lactamase. The present inventors designed the present system to target those areas of interaction for mutagenesis. The structural mimicry suggests that these residues maintain important interactions in the inhibitory complex. The reagents and system disclosed herein may also be used to evaluate the effect each of these amino acids has on the inhibition of TEM-1 β-lactamase and the extended-spectrum-hydrolyzing β-lactamases. The D49A and F142A mutants were used as templates for use in a system for identifying and creating novel antimicrobial agents based on the identification, isolation and selection of inhibitors that are: specific for a specific penicillin binding protein (PBP) or for a broad range of the same. The Kᵢ of each was measured with TEM-1, E104K, and G238S β-lactamases.

Both the D49A and F142A mutants demonstrated an approximate 100 to 300 fold increase in Kᵢ compared to wild-type BLIP when inhibiting TEM-1 β-lactamase. The D49A mutant inhibits TEM-1 with a Kᵢ of 8.29 nM, while the F142A mutant inhibits with a Kᵢ of 33.42 nM. The inhibitory activities of the wild-type, D49A and F142A BLIP inhibitors with the ESBLs show that the BLIP binds E104K in a different manner from that of TEM-1 and the G238S mutant. The Kᵢ values found for D49A and F142A with G238S β-lactamase were similar to the Kᵢ values found for the BLIP mutants binding TEM-1. The D49A BLIP mutant inhibited G238S with a Kᵢ of 9.35 nM. As with TEM-1, the D49A mutation reduced inhibitory activity 100-fold. Likewise, the F142A mutation reduced inhibitory activity approximately 800-fold with a Kᵢ of 54.79 nM for G238S. The fact that these two mutations in BLIP have a similar effect on the Kᵢ values for TEM-1 and G238S β-lactamases shows that BLIP inhibits G238S much in the same way in which it inhibits TEM-1. If either residue were not as important in the inhibition of G238S, then the Kᵢ value for that alanine-mutant would be closer to the wild-type BLIP Kᵢ for G238S. An example where a residue becomes less critical for inhibition of a β-lactamase mutant is position 142 with the E104K B-lactamase.

The Kᵢ of BLIP D49A with E104K is 1.51 pM, which represents a 10-fold increase from wild-type BLIP and E104K. This value suggests that BLIP residue D49 is not as critical to inhibition of E104K as it is to the other enzymes tested. In contrast to what was observed with TEM-1 and G238S, however, there was little change from the wild-type Kᵢ in the BLIP F142A mutant inhibiting E104K (Kᵢ=242.65 nM). The value for F142 does not appear nearly as important for inhibition of E104K as it is for TEM-1 and G238S. BLIP binding to SHV-1 β-lactamase SHV-1 is 68% identical, at the amino acid level, to TEM-1 β-lactamase. How this similarity corresponds to structure is unknown as the crystal structure of SHV-1 β-lactamase has not yet been solved. Using the present invention, therefore, the lack of a crystal structure for each β-lactamase and PBP variant is overcome by isolating a functional protein. While both the TEM-1 and SHV-1 enzymes hydrolyze a similar profile of penicillins and cephalosporins, it is not clear whether the homology between the two enzymes means that BLIP should inhibit both equally well. While it may be predicted that even slight differences in the three-dimensional structure of SHV-1 compared to TEM-1 would effect BLIP binding considerably, the compositions, methods and system disclosed herein solve those problems functionally.

Those issues were addressed by performing an additional inhibitory assay with wild-type BLIP and SHV-1 β-lactamase. SHV-1 was purified to greater than 90% homogeneity (data not shown), and was bound to increasing concentrations of wild-type BLIP. The Kᵢ of BLIP for SHV-1 was found to be 991.7 nM, 9,000-fold higher than what was found for TEM-1.

Table II summarizes the results for the wild-type BLIP and other mutants of BLIP. The inhibitory activities (Kᵢ) for wild-type BLIP and the D49A and F142A mutants with TEM-1, G238S, E104K, and SHV-1 β-lactamase are shown and expressed in nanomoles binding of β-lactamase.

TABLE II

| BLIP | TEM-1 | G238S | E104K | SHV-1 |
|---|---|---|---|---|
| Wild-type | 0.11 ± .001 | 0.07 ± .01 | 138.5 ± 4.5 | 991.7 ± 70.2 |
| D49A | 8.29 ± .63 | 9.35 ± .83 | 1508.6 ± 52.9 | ND |
| F142A | 33.42 ± .51 | 54.79 ± 3.91 | 242.65 ± 15.7 | ND |

ND = Value not determined

The first step toward identifying the amino acids important for BLIP specificity and inhibitory activity was to develop an expression system designed to produce BLIP with wild-type activity. BLIP expressed in its native S. clavuligerus is straightforward and produces large quantities of protein, while expression in another Streptomyces species, S. lividans, produces limited quantities of BLIP. The inventors have now been able to express BLIP in E. coli in order to be able to use protein engineering/selection techniques, such as phage display, to be used. Successful expression of soluble BLIP in E. coli now permits the production, identification and isolation of BLIP mutants. The soluble expressed BLIP and mutants derived as shown herein and using a mutagenesis and selection system as described hereinabove, complements the phage display system by allowing soluble, engineered BLIP mutants to be purified and tested against its target β-lactamase or PBP.

Different methods of expressing BLIP in E. coli, have been attempted with little success. It is not known whether the presence of rare codon, a high GC content (69%), or the requirement for Streptomyces-specific post-translational modification are responsible for this difficulty. An additional possibility is that BLIP itself is toxic to E. coli. Small quantities of BLIP displaying wild-type inhibitory kinetics, however, were purified using a maltose binding-protein fusion system. Optimization of this fusion system was difficult as much protein was lost in the additional purification steps needed to obtain pure BLIP. Histidine-tagged proteins were purified in a relatively simple manner, while usually maintaining the native activity of the tagged-protein. Therefore, an expression system centered around an N-terminal 6xHis-tagged BLIP was constructed. Expression is directed by the inducible trc promoter, and a cat gene is inserted into the plasmid's β-lactamase gene to avoid possible complex formation during purification of BLIP. This system enabled BLIP to be purified to 90% homogeneity in one step.

Calculation of the Kᵢ for BLIP expressed in E. coli was performed using methods derived for tight-binding inhibitors, assuming enzyme and inhibitor interact at a 1:1 stoichiometry. Wild-type his-tagged BLIP was found to have a Kᵢ of 0.11 nM. This value is slightly lower than the previously calculated value of 0.6 nM for BLIP isolated from S. clavuligerous. This difference could be attributed to the manner in which the Kᵢ was calculated, and confirms that the N-terminal his-tag has no effect on BLIP binding. Once wild-type expression of BLIP was achieved, the roles of BLIP residues in the inhibition of different targets may be determined. The crystal structure of BLIP with TEM-1 β-lactamase shows that D49 of BLIP makes strong hydrogen bond contacts with four conserved residues in the TEM-1 active site pocket: S130, K234, S235, and R244. These four amino acids are involved in the binding and catalysis of β-lactam antibiotics and are conserved in all class A β-lactamases. Several, but not all, class A β-lactamases are inhibited by BLIP to various degrees. As a result, the present inventors predicted that D49 may play an important role in inhibition. Mutation of the aspartic acid to an alanine removes the carboxylate moiety that serves as a hydrogen-bond acceptor for the four active site TEM-1 residues. Elimination of the carboxylate dropped the inhibitory activity of BLIP approximately 100-fold, which supports the information yielded by the crystal structure for the BLIP/TEM-1 complex.

The crystal structure also leads to the prediction that F142 is also important for inhibition of TEM-1 β-lactamase. F142 is in contact with β-lactamase residues E104, Y105, N170, A237, G238, and E240 in the inhibitory complex. As in the case of D49, most of these residues are either conserved in class A β-lactamases or are involved in catalysis. Therefore, the contribution F142 makes to inhibition was determined. Mutation of phenylalanine removes the hydrophobic side-chain that mimics the benzyl group in PenG from the TEM-1/PenG complex. The F142 change also decreases inhibition approximately 100-fold, which suggests that the interactions mediated by F142 are important for inhibitor binding, and that they are similar in magnitude to the contributions made by D49.

When attempting to purify mutants that might be degraded by host proteases, adjustments can be made to, for example, the induction conditions for the protein, the temperature of bacterial growth, or the addition of protease inhibitors upon induction of protein production. Alternatively, the protein may be produced in a protease deficient bacterial strain.

While it cannot be ruled out that other amino acids may be tolerated at positions 49 and 142 of BLIP, it was determined that both an aspartic acid at position 49, and a phenylalanine at position 142 are important residues for wild-type levels BLIP inhibitory activity for TEM-1 β-lactamase. The ability of BLIP to inhibit two extended-spectrum β-lactamase mutants was examined, as was the effect of the D49A and F142A mutations on any inhibitory activity observed. The β-lactamase mutation G238S found in TEM-19, and E104K, the mutation found in TEM-17, were the two representative extended-spectrum mutants examined with BLIP and the BLIP mutants. The prevalence of the G238S and E104K substitutions in many of the extended-spectrum β-lactamases makes these two single mutants ideal candidates for targeting.

Interestingly, wild-type BLIP, D49A BLIP, and the F142A BLIP mutants each inhibited G238S at similar levels to which they inhibited wild-type TEM-1 B-lactamase. According to the crystal structure, the only contact made to G238 of TEM-1 is by F142. The fact that no change in the inhibition profile was observed between the β-lactamase enzymes suggests that this contact between G238 and F142 is not critical for wild-type levels of inhibitory activity. If this interaction played a role in BLIP inhibition, then replacement of the glycine side-chain at position 238 of TEM-1 would have resulted in an increased Kᵢ with wild-type BLIP.

In contrast to BLIP and BLIP mutant binding to G238S, significant changes in the inhibitory profile were observed, relative to TEM-1, when E104K was used as the target β-lactamase. Wild-type BLIP inhibited E104K approximately 1000-fold worse than TEM-1, suggesting that the interactions made between BLIP and E104 are critical for wild-type levels of activity. This decrease in activity also rules out the presence of compensatory interactions between BLIP and the lysine at position 104 to restore wild-type levels of inhibition.

The TEM-1 and E104K studies point out that E104 is the amino acid making the most important interaction with the phenylalanine at position 142 of BLIP. The increase in Kᵢ observed when comparing wild-type BLIP to the F142 mutant binding of TEM-1 shows the importance of phenylalanine at this position. Mutation of the glutamic acid at position 104 to lysine in β-lactamase (TEM-17) results in a strong increase in Kᵢ with wild-type BLIP. This result shows that some, or all, of the BLIP residues interacting with E104 (BLIP-E73, K74, F142, and Y 143) are making important interactions. Meanwhile, the F142A BLIP mutation has little effect on the binding E104K as the Kᵢ is less than two-fold greater than the wild-type BLIP K, for E104K. Because the other β-lactamase residues F142 interacts with remain unchanged in the E104K enzyme, it can be concluded that the disruption of the BLIP F142/β-lactamase E104 interaction is what causes the sharp decrease in inhibition. Therefore, in the TEM-1 complex, the loss of inhibitory activity observed with the F142A mutant appears to primarily result from the removal of the F142/E104 contact.

As wild-type BLIP is able to bind extended-spectrum β-lactamases the expression of this, and other mutant proteins in E. coli, aid in the engineering of tighter, smaller inhibitors for these β-lactamases. Protein engineering and selection systems such as the phage display system disclosed herein are easier to develop if the starting protein and target have some degree of affinity prior to the subsequent iterations of mutagenesis and. screening. The present system has also been used to determine which residues are important for binding and for truncating the protein to those that encode epitopes involved in binding. As discussed hereinabove, interactions may be optimized using phage display or a comparable technique.

Determination of the Kᵢ of BLIP with SV-1 β-lactamase shows that even though TEM-1 and SHV-1 are both class A β-lactamases, and are 68% identical, the interactions that make BLIP a tight inhibitor of TEM-1 are not conserved with SHV-1. The system of the invention allows for the determination of interactions when no crystal structure is available, as is the case for SHV-1 and most PBPs. The level of identity between TEM-1 and SHV-1 would suggests that both enzymes share a similar protein fold, however, the present inventors have used the isolation and characterization system disclosed herein to show that the assumption is not correct. The system disclosed herein, however, may be used to determine those differences that are responsible for the discrepancy in Kᵢ. The present system, for example, can be used to determine if a D104E mutation in SHV-1 would improve the Kᵢ of BLIP.

While this invention has been described in reference to illustrative embodiments, this description is not intended to be construed in a limiting sense. Various modifications and combinations of the illustrative embodiments, as well as other embodiments of the invention, will be apparent to persons skilled in the art upon reference to the description. It is therefore intended that the appended claims encompass any such modifications or embodiments.

What is claimed is:

1. A nucleic acid segment encoding a fusion protein, wherein the segment comprises:
a β-lactamase inhibitor protein; and
an affinity carrier forming a fusion protein with said β-lactamase inhibitor protein.

2. The nucleic acid segment of claim 1, wherein said nucleic acid segment further comprises a recombinant vector.

3. The nucleic acid segment of claim 1, wherein said nucleic acid segment further comprises a recombinant expression vector.

4. The nucleic acid segment of claim 1, wherein said nucleic acid segment further comprises an operatively linked promoter.

5. The nucleic acid segment of claim 3, wherein said operatively linked promoter comprises the β-lactamase promoter.

6. The nucleic acid segment of claim 1, wherein said nucleic acid segment further comprises a signal peptide amino from said β-lactamase inhibitor protein.

7. A recombinant host comprising:
a recombinant vector comprising a gene encoding a fusion protein, said fusion protein comprising:
a signal peptide;
a β-lactamase inhibitor protein carboxy from said signal peptide; and
an affinity carrier, wherein said affinity carrier and said β-lactamase inhibitor protein form a fusion protein.

8. The host of claim 7, wherein said recombinant host cell comprises a prokaryotic cell.

9. The host of claim 7, wherein said recombinant host cell comprises an E. coli.

10. The host of claim 7, wherein said recombinant host cell comprises an M13 phage.

11. The host of claim 7, wherein said signal peptide comprises the signal peptide of β-lactamase.

12. The host of claim 7, wherein said recombinant vector further comprises a low-level expression promoter upstream from said gene encoding said fusion protein.

13. The host of claim 12, wherein said low-level expression promoter comprises the β-lactamase promoter.

14. A purified nucleic acid segment encoding a fusion protein, wherein the segment comprises:
a β-lactamase signal sequence;
a β-lactamase inhibitor protein carboxy from said β-lactamase signal sequence; and
a viral coat protein carboxy from said β-lactamase inhibitor protein, wherein said nucleic acid encodes a viral coat protein that forms part of a phage viral coat.

15. The nucleic acid segment of claim 14, wherein said nucleic acid segment further comprises a recombinant vector.

16. The nucleic acid segment of claim 14, wherein said nucleic acid segment further comprises a recombinant expression vector.

17. The nucleic acid segment of claim 14, wherein said nucleic acid segment further comprises an operatively linked promoter.

18. The nucleic acid segment of claim 17, wherein said operatively linked promoter comprises the β-lactamase promoter.

19. A polypeptide composition comprising:
a β-lactamase inhibitor protein; and
an affinity carrier, wherein said affinity carrier is formed as a fusion protein with said β-lactamase inhibitor protein.

20. The polypeptide of claim 19, wherein said affinity carrier comprises g3P.

21. The polypeptide of claim 19, wherein said affinity carrier comprises a maltose binding protein.

22. The polypeptide of claim 19, wherein said affinity carrier comprises a histidine-tag.

23. The polypeptide of claim 19, wherein said polypeptide is expressed in a prokaryotic cell.

24. The polypeptide of claim 19, wherein said polypeptide is expressed in E. coli.

25. A method of isolating an antimicrobial agent comprising the steps of:
displaying a β-lactamase inhibitor protein on a virus;
contacting said virus with a β-lactamase binding protein target;
selecting for the virus that has a higher affinity for the target; and
testing said β-lactamase inhibitor protein for antimicrobial activity.

26. The method of isolating an antimicrobial agent of claim 25, wherein said step of β-lactamase inhibitor protein for antimicrobial activity comprises measuring the growth rate of bacteria to determine if the β-lactamase inhibitor protein is reducing the rate of bacterial cell growth.

27. The method of isolating an antimicrobial agent of claim 25, wherein said step of displaying a β-lactamase inhibitor protein is on an M13 phage.

28. The method of isolating an antimicrobial agent of claim 25, wherein said step of contacting said virus with a β-lactamase binding protein target comprises immobilizing said target to a solid support.

29. The method of isolating an antimicrobial agent of claim 25, wherein said step of contacting said virus with a β-lactamase binding protein target comprises immobilizing said target to oxirane beads.

30. The method of isolating an antimicrobial agent of claim 25, wherein said step of selecting for the virus that has a higher affinity for the target further comprises the steps of:
washing said β-lactamase binding protein phage-target complex in a buffer comprising a pH buffered salt solution at about physiologic pH; and
eluting said β-lactamase binding protein phage.

31. The method of isolating an antimicrobial agent of claim 30, wherein said step of eluting said β-lactamase binding protein phage occurs under conditions that do not significantly affect phage viability.

32. The method of isolating an antimicrobial agent of claim 25, wherein said step of testing said β-lactamase inhibitor protein for antimicrobial activity is further defined as comprising the steps of:
contacting the isolated antimicrobial agent with a bacterium; and
measuring the viability of the bacterium after a predetermined period of time sufficient to determine said viability.

33. A system of identifying, selecting and improving an antimicrobial agent comprising the steps of:
(a) creating a mutant β-lactamase inhibitor protein phage display library;
(b) selecting mutant β-lactamase inhibitor protein phage by comparing one or more characteristics of the mutant β-lactamase inhibitor display phage;
(c) cloning the selected mutant β-lactamase inhibitor protein phage;
(d) conducting mutagenesis on the selected mutant β-lactamase inhibitor protein phage to create a new mutant β-lactamase inhibitor protein phage display:
(d1) evaluating the performance of each β-lactamase inhibitor protein phage by panning for those having a high affinity for a binding target,
(d2) eliminating β-lactamase inhibitor protein phage whose performance is less than a specified performance level, and
(d3) selecting mutants from the mutant β-lactamase inhibitor protein phage, each mutants of mutant β-lactamase inhibitor protein phage having antimicrobial performance that is equal to or greater than the specified performance level; and
(e) repeating steps (b) and (c).

34. The system of claim 33, wherein the step of cloning the mutant β-lactamase inhibitor protein phage further includes determining the nucleic acid sequence of the mutant β-lactamase inhibitor protein.

35. The system of claim 33, further comprising the step of truncating the mutant β-lactamase inhibitor protein prior to repeating steps (b) and (c).

36. The system of claim 33, wherein said binding target used in the step of evaluating the performance of each β-lactamase inhibitor protein phage by panning for those having a high affinity for a binding target comprises one or more β-lactamases.

37. The system of claim 33, wherein said binding target used in the step of evaluating the performance of each β-lactamase inhibitor protein phage by panning for those having a high affinity for a binding target comprises one or more penicillin binding proteins.

38. The system of claim 33, wherein the step of eliminating β-lactamase inhibitor protein phage whose performance is less than a specified performance level is further defined as comprising the steps of:
isolating the β-lactamase inhibitor protein displayed on the phage to obtain an antimicrobial agent;
contacting said isolated antimicrobial agent with a bacterium; and
measuring the viability of the bacterium after a predetermined period of time sufficient to determine said viability.
